(19) United States
(12) Patent Application Publication
(10) Pub. No.: US 2014/0242180 A1
(43) Pub. Date: Aug. 28, 2014

(54) NANOPARTICLES FOR CONTROLLING BLEEDING AND DRUG DELIVERY

(71) Applicant: CASE WESTERN RESERVE UNIVERSITY, Cleveland, OH (US)

(72) Inventors: Erin Lavik, Cleveland Heights, OH (US); Andrew Shoffstall, Cleveland Heights, OH (US); Jeffrey Ustin, Cleveland, OH (US)

(21) Appl. No.: 14/351,807

(22) PCT Filed: Oct. 12, 2012
(86) PCT No.: PCT/US2012/060003

§ 371 Date: Apr. 14, 2014

Related U.S. Application Data
(60) Provisional application No. 61/546,826, filed on Oct. 13, 2011.

(51) Int. Cl. A61K 9/14; A61K 47/48

(52) U.S. Cl. CPC A61K 9/14; A61K 47/48215; A61K 47/482
USPC 424/499; 525/54.1; 530/331; 424/78.17; 514/13.9; 428/402

(57) ABSTRACT

A temperature stable nanoparticle is provided comprising a core, a water soluble polymer and a peptide, the water soluble polymer attached to the core at a first terminus of the water soluble polymer, the peptide attached to a second terminus of the water soluble polymer, the peptide comprising an RGD amino acid sequence, the water soluble polymer of having sufficient length to allow binding of the peptide to glycoprotein lib/Ilia (GPIIb/llla). In one aspect, the nanoparticle has a melting temperature over 35° C. In various aspects, the nanoparticle has a spheroid shape and a diameter of less than 1 micron.

This application claims priority of U.S. Provisional Patent Application No. 61/564826, filed Oct. 13, 2011, the disclosure of which is incorporated by reference in its entirety.

### STATEMENT OF GOVERNMENT INTEREST

This invention was made with government support under Grant Number 1DP20D007338-01 awarded by the National Institutes of Health and Grant Number W81XWH-11-2-0014 awarded by the United States Department of Defense. The government has certain rights in the invention.

### BACKGROUND

Normally, when an injury occurs, platelets become activated at the injury site and the activated platelets produce fibrin and the cells and fibrin form a plug that halts bleeding (5). In uncontrolled bleeding, the platelets are not able to form a plug. There are a number of approaches to augment hemostasis in the field and clinic including pressure dressings, absorbent materials such as QuikClot®, and intravenous (IV) infusion of activated recombinant factor VII (rFVIIa), but the former two are only applicable to exposed wounds, and rFVIIa has had both mixed results, requires refrigeration, and is exceptionally expensive making it challenging to administer in the field or at the site of trauma. Clearly, a new approach to halt bleeding that is amenable to administration in the field is needed.

Hemorrhaging is also the first step in the injury cascade, for example, in the central nervous system (CNS). In both spinal cord and traumatic brain injuries, the first observable phenomena, regardless of mechanism of insult, is hemorrhaging. If one can stop the bleeding, presumably one can preserve tissue and improve outcomes. The primary mechanical insult is very often a small part of the injury. The secondary injury processes that occur over hours, days, and weeks following injury lead to progression and the poor functional outcomes. Stopping those secondary injury processes would mean preservation of greater amounts of tissue. Preservation of tissue means better functional outcomes.

Following injury, hemostasis is established through a series of coagulatory events. The critical steps in terms of platelets involve their activation, binding, and release of a host of growth factors and other molecules including fibrinogen. During vascular injury, collagen is exposed which triggers the activation of platelets. Platelet morphology shifts from a discoid to stellate, and they adhere to the exposed collagen. Once platelet aggregation begins, several inflammatory agents are released from their storage granules including adenosine diphosphate (ADP), which causes the surfaces of nearby circulating platelets to become adherent. Serotonin, epinephrine, and thromboxane A 2 further induce extreme vasoconstriction. The ultimate step, clot formation, is the conversion of fibrinogen, a large, soluble plasma protein produced by the liver and normally present in the plasma, into fibrin, an insoluble, threadlike molecule.

In severe injuries, these endogenous processes fall short and uncontrolled bleeding results. There have been a number approaches to augment these processes and induce hemostasis beyond the external methods. Platelet substitutes which either replace or augment the existing platelets have been pursued for a number of years (6). Administration of allogeneic platelets can help to halt bleeding; however, platelets have a short shelf life, and administration of allogeneic platelets can cause graft versus host disease, alloimmunization, and transfusion-associated lung injuries (6). Non-platelet alternatives including red blood cells modified with the Arg-Gly-Asp (RGD) sequence, fibrinogen-coated microcapsules based on albumin, and liposomal systems have been studied as coagulants (7), but toxicity, thrombosis, and limited efficacy are major issues in the clinical application of these products (8).

Recombinant factors including rFVIIa (NovoSeven®) can augment hemostasis by promoting the production of fibrinogen, but immunogenic and thromboembolic complications are unavoidable risks (9). Nevertheless, NovoSeven® is being used in the clinic in a number of trauma and surgical situations where bleeding cannot otherwise be controlled (9). The data on its efficacy is variable, but it cannot be that NovoSeven is exceedingly expensive. A single dose costs approximately $10,000, and multiple doses are typically needed to impact hemostasis (9).

For a hemostat to be effective for complex trauma, the system needs to be non-toxic, stable when stored at room temperature (i.e. a medic's bag), have the potential for immediate I.V. administration, and possess injury site-specific aggregation properties so as to avoid non-specific thrombosis. For this system to be clinically translatable, ideally it needs to be made with materials previously approved by the FDA. Practically, it also needs to be affordable.

### SUMMARY OF THE INVENTION

A temperature stable nanoparticle is provided comprising a core, a water soluble polymer and a peptide, the water soluble polymer attached to the core at a first terminus of the water soluble polymer, the peptide attached to a second terminus of the water soluble polymer, the peptide comprising an RGD amino acid sequence, the water soluble polymer of having sufficient length to allow binding of the peptide to glycoprotein IIb/IIIa (GPIIb/IIIa). In one aspect, the nanoparticle has a melting temperature over 35° C. In various aspects, the nanoparticle has a spheroid shape and a diameter of less than 1 micron.

In various aspects, the nanoparticle has a diameter between 0.1 micron and 1 micron.

In various aspects, the nanoparticle is non-spheroid, a rod, fiber or whisker. In various embodiments of this aspect, nanoparticle has an aspect ratio length to width of at least 3.

In various aspects, the nanoparticle is stable at room temperature for at least 14 days.

A plurality of nanoparticles, is also provided, wherein each nanoparticle as provide by the disclosure, has an average diameter between 0.1 micron and 1 micron.

In various aspects of the plurality of nanoparticles, greater than 75% of all nanoparticles have a diameter between 0.1 micron and 1 micron.

In various aspects, the nanoparticle of the disclosure has a core that is a crystalline polymer, a single polymer, a block copolymer, a triblock copolymer or a quadblock polymer. In various aspect, the core comprises PLGA, PLA, PGA, (poly (ε-caprolactone) PCL, PLL or combinations thereof.

In various aspects, the nanoparticle core is biodegradable, solid, non-biodegradable and/or comprised of a material selected from the group consisting of gold, silver, platinum, aluminum, palladium, copper, cobalt, indium, nickel, ZnS, ZnO, Ti, TiO₂, Sn, SnO₂, Si, SiO₂, Fe, Fe⁺⁴, steel, cobalt-chrome alloys, Cd, CdSe, CdS, and CdS, titanium alloy, AgI, AgBr, HgI₂, PbS, PbSe, ZnTe, CdTe, In₂S₃, In₂Se₃, Cd₃P₂, Cd₃As₂, InAs, GaAs, cellulose or a dendrimer structure.

In various aspects, the water soluble polymer in the nanoparticle is selected from the group consisting of polyethylene glycol (PEG), branched PEG, polysialic acid (PSA), carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG), polyoxazoline, poly acryloylmorpholine, polyvinyl alcohol (PVA), polycarboxylate, polyvinylpyrrolidone, polyphosphazene, polyoxazoline, polyethylene-co-maleic acid anhydride, polystyrene-co-maleic acid anhydride, poly(1-hydroxymethylethylene hydroxymethylformal) (PHF), 2-methacryloyloxy-2′-ethyltrimethylammoniumphosphate (MPC), polyethylene glycol propionaldehyde, copolymers of ethylene glycol/propylene glycol, monomethoxy-polyethylene glycol, carboxymethylcellulose, polyacetals, poly-1,3-dioxolane, poly-1,3,6-trioxane, ethylene/maleic anhydride copolymer, poly (β-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll or dextran and combinations or mixtures thereof. In various aspects, the water soluble polymer is PEG having an average molecular weight between 100 Da and 10,000 Da or at least about 100.

In various aspects, the peptide of the nanoparticle comprises a sequence selected from the group consisting of RGD, RGDS, GRGDS, GRGDSP, GRGDSPK, GRGDN, GRGDNP, GGGGRGDS, GRGDK, GRGDTP, cRGD, YRGDS or variants thereof. In various aspects, the peptide is linear and in other aspects, the peptide is cyclic. A cyclic peptide is understood in the art to include those that are cyclic as a result of covalent association, and those that are cyclic by virtue of a conformation preference. Accordingly, cyclic peptides include those that are not cyclic through covalent bonding. In various aspects, the RGD peptide is in a tandem repeat. In various aspects, the nanoparticle comprises 1, 2, 3, 4, 5, 6, 7, 8, 9, 10 or more copies of the RGD peptide, or multiple copies of the RGD peptide. In various aspects, all of the RGD peptides are in the nanoparticle are the same, and in other aspects, two copies of the RGD peptide have different sequences.

In various aspects, the water soluble polymer is attached to the core at a molar ratio of 0.1:1 to 1:10 or greater.

In various aspects, the nanoparticle of the disclosure further comprising a therapeutic compound. In various aspects, the therapeutic compound is hydrophobic, the therapeutic compound is hydrophilic, the therapeutic compound is covalently attached to the nanoparticle, non-covalently associated with the nanoparticle, associated with the nanoparticle through electrostatic interaction, or associated with the nanoparticle through hydrophobic interaction, the therapeutic compound is a growth factor, a cytokine, a steroid, or a small molecule, and/or the therapeutic compound is a anti-cancer compound.

A pharmaceutical composition comprising the nanoparticle of the disclosure is provided. In various aspects, the pharmaceutical composition is an intravenous administration formulation, a lyophilized formulation, or a powder.

A method of treating an condition in an individual is also provided comprising the step of administering the nanoparticle of the disclosure to a patient in need thereof in an amount effective to treat the condition. In various aspects, the individual has a bleeding disorder. In various aspects of the method, the nanoparticle is administered in an amount effective to reduce bleeding time by more than 15% compared to no administration or administration of saline. In various aspects, the bleeding disorder is a symptom of a clotting disorder, thrombocytopenia, a wound healing disorder, trauma, blast trauma, a spinal cord injury or hemorrhaging.

### DESCRIPTION OF THE INVENTION

A functionalized nanoparticle is provided based on FDA-approved materials that has multiple uses. In various aspects, the nanoparticle reduces bleeding time at the site of injury, plays a role in hemostasis following trauma to the central nervous system (CNS) and provides a means for localized drug delivery.

Nanoparticles are provided based on a polymer core, a water soluble polymer, and a variant on the arginine-glycine-aspartic acid (RGD) moiety.

### I. Nanoparticle

The disclosure provides a nanoparticle comprising a core, a water soluble polymer and a peptide, the water soluble polymer attached to the core at a first terminus of the water soluble polymer, the peptide attached to a second terminus of the water soluble polymer, the peptide comprising an RGD amino acid sequence, the water soluble polymer of having sufficient length to allow binding of the peptide to glycoprotein IIb/IIIa (GPIIb/IIIa). In various aspects, the peptide is linear or cyclic. It will be appreciated that in a composition comprising a plurality of nanoparticles of the disclosure, the composition is contemplated to include nanoparticles wherein all peptides are linear, all peptides are cyclic, or a mixture of linear and cyclic peptides is present.

Nanoparticles of the disclosure are temperature stable in that they maintain essentially the same structure and/or essentially the same function over a wide range of temperatures. By “essentially the same structure” and “essentially the same function,” the disclosure contemplates “essentially the same” to mean without a change that affects the ability of the nanoparticles to carry out its use at a dosage of plus or minus 10% of an original dosage, plus or minus 10% of an original dosage, plus or minus 10% of an original dosage, plus or minus 9% of an original dosage, plus or minus 8% of an original dosage, plus or minus 7% of an original dosage, plus or minus 6% of an original dosage, plus or minus 5% of an original dosage, or plus or minus 5%-10% of an original dosage. In various embodiments, the nanoparticles maintain essentially the same structure and/or essentially the same function at physiological temperature, regardless of the temperature at which the nanoparticles were produced. Nanoparticles that maintain essentially the same structure and/or essentially the same function at temperatures elevated well over physiological temperatures are also contemplated. The ability to maintain essentially the same structure and/or essentially the same function at elevated temperatures is important for any number of reasons, including, for example and without limitation, sterilization processes. On the other hand, nanoparticles which maintain essentially the same structure and/or essentially the same function at reduced temperatures are also contemplated. For example, nanoparticles that maintain essentially the same structure and/or essentially the same function at or below freezing temperatures are contemplated for formulations that require or benefit from long term storage. In various aspects the nanoparticle of the disclosure have a melting temperature over 35° C., over 40° C., over 45° C., over 50° C., over 55° C., over 60° C., over 65° C., over 70° C., over 71° C., over 72° C., over 73° C., over 74° C., over 75° C., over 76° C., over 77° C., over 78° C., over 79° C. or over 80° C.

The nanoparticle of all aspects of the disclosure are stable at room temperature for at least 5 days, at least 6 days, at least 7 days, at least 8 days, at least 9 days, at least 10 days, at least 11 days, at least 12 days, at least 13 days or at least 14 days or more.

Nanoparticle of the disclosure are contemplated to have any of a number of different shapes. The shape of the nanoparticle is in certain aspects, a function of the method of its production. In other aspects, the nanoparticle acquires a shaped that is formed before, during or after the process of its production. In various embodiments, nanoparticles are provided that have a spheroid shape. Spheroid nanoparticles (referred to herein as nanospheres) having various sizes are contemplated, wherein, for example nanoparticles having a diameter between 0.1 micron and 0.5 micron, between 0.2 micron and 0.4 micron, between 0.25 micron and 0.375 micron, between 0.3 micron and 0.375 micron, between 0.325 micron and 0.375 micron, between 0.12 microns and 0.22 microns, between 0.13 microns and 0.22 microns, between 0.14 microns and 0.22 microns, between 0.15 microns and 0.22 microns, between 0.16 microns and 0.22 microns, between 0.17 microns and 0.22 microns, between 0.18 microns and 0.22 microns, between 0.19 microns and 0.22 microns, between 0.20 microns and 0.22 microns, between 0.21 microns and 0.22 microns, between 0.12 microns and 0.21 microns, between 0.12 microns and 0.20 microns, between 0.12 microns and 0.19 microns, between 0.12 microns and 0.18 microns, between 0.12 microns and 0.17 microns, between 0.12 microns and 0.16 microns, between 0.12 microns and 0.15 microns, between 0.12 microns and 0.14 microns, or between 0.12 microns and 0.13 microns are contemplated. In various aspect, nanoparticles are contemplated having a diameter of 0.01 microns to 1.0 micron, 0.05 microns to 1.0 micron, 0.05 microns to 0.95 microns, 0.05 microns to 0.9 microns, 0.05 microns to 0.85 microns, 0.05 microns to 0.8 microns, 0.05 microns to 0.75 microns, 0.05 microns to 0.7 microns, 0.05 microns to 0.65 microns, 0.05 microns to 0.6 microns, 0.05 microns to 0.55 microns, 0.05 microns to 0.5 microns, 0.1 microns to 1 micron, 0.15 microns to 1.0 microns, 0.2 microns to 1 micron, 0.25 microns to 1.0 microns, 0.3 microns to 1 micron, 0.35 microns to 1.0 microns, 0.4 microns to 1 micron, 0.45 microns to 1.0 microns, or 0.5 microns to 1 micron. In compositions of nanoparticles provided by the disclosure, the spherical nanoparticles are homogenous in that that all have the same diameter, or they are heterogeneous in that at least two nanoparticles in the composition have different diameters.

Nanoparticle are also provided which are non-spheroid. Other nanoparticles include those having a rod, fiber or whisker shape. In rod, fiber or whisker embodiments, the nanoparticle has a sufficiently high aspect ratio to avoid, slow or reduce the rate of clearance from circulation.

Aspect ratio is a term understood in the art, a high aspect ratio indicates a long and narrow shape and a low aspect ratio indicates a short and thick shape.

Nanoparticle of the disclosure are contemplated with an aspect ratio length to width of at least 3, of at least 3.5, of at least 4.0, of at least 4.5, of at least 5.0, of at least 5.5, of at least 6.0, of at least 6.5, of at least 7.0, of at least 7.5, of at least 8.0, of at least 8.5, of at least 9.0, of at least 9.5, of at least 10.0 or more. In a composition of nanoparticles contemplated, the nanoparticles have, in one embodiment, identical aspect ratios, and in alternative embodiments, at least two nanoparticles in the composition have different aspects ratios. Composition of nanoparticles are also characterized by having, on average, essentially the same aspect ratio. “Essentially the same” as used in this instance indicated that variation in aspect ratio of about 10%, about 9%, about 8%, about 7% about 6% or up to about 5% is embraced. In still other aspects, a composition of nanoparticles is provided wherein the nanoparticles in the composition have an aspect ratio of between about 1% and 200%, between about 1% and 150%, between about 1% and 100%, between about 1% and about 50%, between about 50% and 200%, between about 100% and 200%, and between about 150% and 200%. Alternatively, the nanoparticles in the composition have an aspect ratio from about X% to Y%, wherein X from 1 up to 100 and Y is from 100 up to 200.

The disclosure also provides a plurality of nanoparticles. In compositions comprising a plurality of spherical nanoparticles provided by the disclosure, nanoparticles in the plurality have an average diameter between 0.1 micron and 0.5 micron, between 0.2 micron and 0.4 micron, between 0.25 micron and 0.375 micron, between 0.3 micron and 0.375 micron, between 0.325 micron and 0.375 micron, about 0.12 micron, about 0.13 micron, about 0.14 micron, about 0.15 micron, about 0.16 micron, about 0.17 micron, about 0.18 micron, about 0.19 micron, about 0.20 micron, about 0.21 micron, about 0.22 micron, about 0.23 micron, about 0.24 micron, about 0.25 micron, about 0.26 micron, about 0.27 micron, about 0.28 micron, about 0.29 micron, about 0.30 micron, about 0.31 micron, about 0.32 micron, about 0.33 micron, about 0.34 micron, about 0.35 micron, about 0.36 micron, about 0.37 micron, about 0.38 micron, about 0.39 micron, about 0.40 micron, about 0.41 micron, about 0.42 micron, about 0.43 micron, about 0.44 micron, about 0.45 micron, about 0.46 micron, about 0.47 micron, about 0.48 micron, about 0.49 micron, about 0.50 micron, about 0.41 micron, about 0.52 micron, about 0.53 micron, about 0.54 micron, about 0.55 micron, about 0.56 micron, about 0.57 micron, about 0.58 micron, about 0.59 micron, about 0.60 micron, about 0.61 micron, about 0.62 micron, about 0.63 micron, about 0.64 micron, about 0.65 micron, about 0.66 micron, about 0.67 micron, about 0.68 micron, about 0.69 micron, about 0.70 micron, about 0.71 micron, about 0.72 micron, about 0.73 micron, about 0.74 micron, about 0.75 micron, about 0.76 micron, about 0.77 micron, about 0.78 micron, about 0.79 micron, about 0.80 micron, about 0.81 micron, about 0.82 micron, about 0.83 micron, about 0.84 micron, about 0.85 micron, about 0.86 micron, about 0.87 micron, about 0.88 micron, about 0.89 micron, about 0.90 micron, about 0.91 micron, about 0.92 micron, about 0.93 micron, about 0.94 micron, about 0.95 micron, about 0.96 micron, about 0.97 micron, about 0.98 micron, about 0.99 micron, about 1.0 micron ,or more.

In various aspects, the plurality of spherical nanoparticles are characterized in that greater than 75%, greater than 80%, greater than 85%, greater than 90%, greater than 95%, greater than 96%, greater than 97%, greater than 98%, or greater than 99% of all nanoparticles have a diameter between 0.1 micron and 0.5 micron, between 0.2 micron and 0.4 micron, between 0.25 micron and 0.375 micron, between 0.3 micron and 0.375 micron, between 0.325 micron and 0.375 micron, between 0.12 microns and 0.22 microns, between 0.13 microns and 0.22 microns, between 0.14 microns and 0.22 microns, between 0.15 microns and 0.22 microns, between 0.16 microns and 0.22 microns, between 0.17 microns and 0.22 microns, between 0.18 microns and 0.22 microns, between 0.19 microns and 0.22 microns, between 0.20 microns and 0.22 microns, between 0.21 microns and 0.22 microns, between 0.12 microns and 0.21 microns, between 0.12 microns and 0.20 microns, between 0.12 microns and 0.19 microns, between 0.12 microns and 0.18 microns, between 0.12 microns and 0.17 microns, between 0.12 microns and 0.16 microns, between 0.12 microns and 0.15 microns, between 0.12 microns and 0.14 microns, between 0.12 microns and 0.13 microns, 0.01 microns to 1.0 micron, 0.05 microns to 1.0 micron, 0.05 microns to 0.95 microns, 0.05 microns to 0.9 microns, 0.05 microns to 0.85 microns, 0.05 microns to 0.8 microns, 0.05 microns to 0.75 microns, 0.05 microns to 0.7 microns, 0.05 microns to 0.65 microns, 0.05 microns to 0.6 microns, 0.05 microns to 0.55 microns, 0.05 microns to 0.5 microns, 0.1 microns to 1 micron, 0.15 microns to 1.0 microns, 0.2 microns to 1 micron, 0.25 microns to 1.0 microns, 0.3 microns to 1 micron, 0.35 microns to 1.0 microns, 0.4 microns to 1 micron, 0.45 microns to 1.0 microns, or 0.5 microns to 1 micron.

The disclosure further provides nanoparticles of essentially any shape are formed using microfabrication processes well known and routinely practiced in the art. In microfabrication methods, size and shape of the nanoparticles are predetermined by design.

In aspects wherein the nanoparticles are utilized which are non-spherical in shape, nanofabrication techniques well known and routinely used in the art are contemplated for production. Daum et al., (2012) Wiley Interdiscip Rev Nanomed Nanobiotechnol 4: 52-65; Gang et al., (2011) ACS Nano 5: 8459-8465; Grilli et al., (2011) Proc Natl Acad Sci U S A 108: 15106-15111; Lin, et al., (2011) Control Release 154: 84-92; Slingenbergh et al., (2012) Selective Functionalization of Tailored Nanostructures. ACS Nano. Molds are produced out of materials such as silicon, PDMS (polydimethylsiloxane) or other materials well known in the art, and cast with a hydrogel such as gelatin. Any polymer as described herein is used to cast the nanoparticles. The resulting structures, based on the original mold are, in various aspects, multiarmed stars with arm lengths from 200 nm to several microns and arm diameters from 200 nm to several microns. Because it is a casting procedure, the casting process allows arms to be of different lengths and dimensions from 3 arms to tens of arms.

### A. Core

A nanoparticle as described above is provided wherein the core is a polymer. In various aspects, the core is a crystalline polymer. “Crystalline” as used herein and understood in the art is defined to mean an arrangement of molecules in regular three dimensional arrays. In other aspects, the polymers are semi-crystalline which contain both crystalline and amorphous regions instead of all molecule arranged in regular three dimensional arrays. In various aspects, the core is a single polymer, a block copolymer, or a triblock copolymer. In specific aspects, the core comprises PLGA, PLA, PGA, (poly (c-caprolactone) PCL, PLL, cellulose, poly(ethylene-co-vinyl acetate), polystyrene, polypropylene, dendrimer-based polymers or combinations thereof.

In various aspects, the core is biodegradable or non-biodegradable, or in a plurality of nanoparticles, combinations of biodegradable and non-biodegradable cores are formulated in contemplated. In various aspects, the core is solid, porous or hollow. In pluralities of nanoparticles, it is envisioned that mixtures of solid, porous and/or hollow cores are included..

Nanoparticle of any aspect of the disclosure include those wherein the core alternatively is a material selected from the group consisting of gold, silver, platinum, aluminum, palladium, copper, cobalt, indium, nickel, ZnS, ZnO, Ti, TiO₂, Sn, SnO₂, Si, SiO₂, Fe, Fe⁺⁴, steel, cobalt-chrome alloys, Cd, CdSe, CdS, and CdS, titanium alloy, AgI, AgBr, HgI₂, PbS, PbSe, ZnTe, CdTe, In₂S₃, In₂Se₃, Cd₃P₂, Cd₃As₂, InAs, GaAs, cellulose or a dendrimer structure.

Hydrogel core are also provided. In one aspect, the hydrogel core provides a higher degree of temperature stable, be less likely to shear vessels and induce non-specific thrombosis and allow formation of larger nanoparticles.

### B. Water Soluble Polymer

A nanoparticle of the disclosure is provided wherein the water soluble polymer is selected from the group consisting of polyethylene glycol (PEG), branched PEG, polysialic acid (PSA), carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG), polyoxazoline, poly acryloylmorpholine, polyvinyl alcohol (PVA), polycarboxylate, polyvinylpyrrolidone, polyphosphazene, polyoxazoline, polyethylene-co-maleic acid anhydride, polystyrene-co-maleic acid anhydride, poly(1-hydroxymethylethylene hydroxymethylformal) (PHF), 2-methacryloyloxy-2′-ethyltrimethylammoniumphosphate (MPC), polyethylene glycol propionaldehyde, copolymers of ethylene glycol/propylene glycol, monomethoxy-polyethylene glycol, carboxymethylcellulose, polyacetals, poly-1,3-dioxolane, poly-1,3,6-trioxane, ethylene/maleic anhydride copolymer, poly (β-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll or dextran and combinations or mixtures thereof. In a plurality of nanoparticles contemplated by the disclosure, each nanoparticle is contemplated, in various aspects, to have the same water soluble polymer, or alternatively, at least two nanoparticles in the plurality each have a different water soluble polymer attached thereto.

In a specific aspect, the nanoparticle of the disclosure is one wherein the water soluble polymer is PEG. For nanoparticles in this aspect, the PEG has an average molecular weight between 100 Da and 10,000 Da, 500 Da and 10,000 Da, 1000 Da and 10,000 Da, 1500 Da and 10,000 Da, 2000 Da and 10,000 Da, 2500 Da and 10,000 Da, 3000 Da and 10,000 Da, 3500 Da and 10,000 Da, 4000 Da and 10,000 Da, 4500 Da and 10,000 Da, 5000 Da and 10,000 Da, 5500 Da and 10,000 Da, 1000 Da and 9500 Da, 1000 Da and 9000 Da, 1000 Da and 8500 Da, 1000 Da and 8000 Da, 1000 Da and 7500 Da, 1000 Da and 7000 Da, 1000 Da and 6500 Da, or 1000 Da and 6000 Da..Alternatively, the nanoparticle is one in which PEG has an average molecular weight of about 100, Da, 200 Da, 300 Da, 400 Da, 1000 Da, 1500 Da, 3000 Da, 3350 Da, 4000 Da, 4600 Da, 5,000 Da, 8,000 Da, or 10,000 Da. In a plurality of nanoparticles, it is contemplated that each nanoparticle is attached to a PEG water soluble polymer of the same molecular weight, or in the alternative, at least two nanoparticles in the plurality are each attached to a PEG water soluble polymer which do not have the same molecular weight.

The nanoparticle of the disclosure includes those wherein the water soluble polymer is attached to the core at a molar ratio of 0.1:1, 0.2:1, 0.3:1, 0.4:1, 0.5:1, 0.6:1, 0.7:1, 0.8:1, 0.9:1, 1:1, 1:2, 1:3, 1:4, 1:5, 1:6, 1:7, 1:8, 1:9, 1:10 or greater. In various aspect, a plurality is proved wherein the water soluble polymer to0 core ratio is identical for each nanoparticle in the plurality, and in alternative aspect, at least two nanoparticles in the plurality have different water soluble polymer to core ratios.

The degree to which a nanoparticle is associated with a water soluble polymer is, in various aspects, determined by the route of administration chosen.

### C. Peptide

The nanoparticle of the disclosure is characterized by having a peptide associated therewith. In various aspects of the disclosure. The peptide is linear or cyclic. In specific embodiments, the peptide comprises a core sequence selected from the group consisting of RGD, RGDS, GRGDS, GRGDSP, GRGDSPK, GRGDN, GRGDNP, GGGGRGDS, GRGDK, GRGDTP, cRGD, YRGDS or variants thereof. Variants are used herein include peptides have a core sequence as defined herein and one or more additional amino acid residues attached at one or both ends of the core sequence, a peptide having a core sequence as defined herein but wherein one or more amino acid residues in the core sequence is substituted with an alternative amino acid residue; the alternative amino acid residue being a naturally-occurring amino acid residue or a non-naturally-occurring amino acid residue, a peptide having a core sequence as defined herein but wherein one or more amino acid residues in the core sequence is deleted, or combinations thereof, wherein the additional amino acid residue, the amino acid substitution, the amino acid deletion or the combination of changes does (or do) not essentially alter the activity of the nanoparticle. “Essentially” as used in this aspect is the same as the meaning described elsewhere in the disclosure.

In various aspects, the RGD peptide is in a tandem repeat arrangement and in embodiments of this aspects, 1, 2, 3, 4, 5, 6, 7, 8, 9, 10 or more copies of the RGD peptide are contemplated. In another aspect, multiple copies of an RGD peptide are attached to the same nanoparticle, albeit not in a random repeat arrangement.

In various aspects wherein the nanoparticle is associated with multiple RGD peptides, the disclosure provide a nanoparticle wherein all copies of the RGD peptide are the same, as wells as aspects wherein two of the RGD peptide have different sequences.

In a plurality of nanoparticles contemplated, embodiments are provided wherein the RGD peptide (or multiple copies of RGD peptides) are identical on each nanoparticle in the plurality. In alternative aspects, at least two nanoparticles in the plurality each are associated with one or more distinct RGD peptides.

In various aspect, the number of peptides on a nanoparticle, i.e., the peptide density, affects platelet aggregation.

E. Other Compounds with the Nanoparticle

A nanoparticle of the disclosure is also contemplated further comprising a therapeutic compound. In various aspects, the therapeutic compound is hydrophobic and in still other aspects, the therapeutic compound is hydrophilic. A nanoparticle of the disclosure is provided wherein the therapeutic compound is covalently attached to the nanoparticle, non-covalently associated with the nanoparticle, associated with the nanoparticle through electrostatic interaction, or associated with the nanoparticle through hydrophobic interaction. In various embodiments, the therapeutic compound is a growth factor, a cytokine, a steroid, or a small molecule. Embodiments are contemplated wherein more than one therapeutic compound is associated with a nanoparticle. In this aspect, each therapeutic compounds associated with the nanoparticle is the same, or each therapeutic compound associated with the nanoparticle is different. In a plurality of nanoparticles provided by the disclosure, each nanoparticle in the plurality is associated with the same therapeutic compound or compounds, or in the alternative, at least two nanoparticles in the plurality is each associated with one or more different therapeutic compounds.

In various aspects, the therapeutic compound is a anti-cancer compound, and in specific embodiments, the therapeutic compound is selected from the group consisting of : an alkylating agents including without limitation nitrogen mustards, such as mechlor-ethamine, cyclophosphamide, ifosfamide, melphalan and chlorambucil; nitrosoureas, such as without limitation carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as without limitation busulfan; triazines such as dacarbazine (DTIC); antimetabolites including folic acid analogs such as methotrexate and trimetrexate; pyrimidine analogs such as without limitation 5-fluorouracil, fluorodeoxyuridine, gemcitabine, cytosine arabinoside (AraC, cytarabine), 5-azacytidine, 2,2′-difluorodeoxycytidine; purine analogs such as without limitation 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including without limitation antimitotic drugs such as paclitaxel; vinca alkaloids including without limitation vinblastine (VLB), vincristine, and vinorelbine, taxotere, estramustine, and estramustine phosphate; epipodophylotoxins such as without limitation etoposide and teniposide; antibiotics such as without limitation actimomycin D, daunomycin (rubidomycin), doxorubicin, mitoxantrone, idarubicin, bleomycins, plicamycin (mithramycin), mitomycinC, and actinomycin; enzymes such as without limitation L-asparaginase; biological response modifiers such as without limitation interferon-alpha, IL-2, G-CSF and GM-CSF; miscellaneous agents including without limitation platinum coordination complexes such as cisplatin and carboplatin; anthracenediones such as without limitation mitoxantrone; substituted urea such as without limitation hydroxyurea; methylhydrazine derivatives including without limitation N-methylhydrazine (MIH) and procarbazine; adrenocortical suppressants such as without limitation mitotane (o,p′-DDD) and aminoglutethimide; hormones and antagonists including without limitation adrenocorticosteroid antagonists such as prednisone and equivalents, dexamethasone and aminoglutethimide; progestin such as without limitation hydroxyprogesterone caproate, medroxyprogesterone acetate and megestrol acetate; estrogen such as without limitation diethylstilbestrol and ethinyl estradiol equivalents; antiestrogen such as without limitation tamoxifen; androgens including testosterone propionate and fluoxymesterone/equivalents; antiandrogens such as without limitation flutamide, gonadotropin-releasing hormone analogs and leuprolide; non-steroidal antiandrogens such as without limitation flutamide; folate inhibitors; tyrosine kinase inhibitors such as without limitation AG1478, and radiosensitizing compounds.

In various aspects, the therapeutic compound is selected from the group consisting of AG1478, acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bicalutamide, bisantrene, bisnafide dimesylate, bizelesin, bleomycin, brequinar, bropirimine, busulfan, cactinomycin, calusterone, capecitabine, caracemide, carbetimer, carboplatin, carmustine, carubicin, carzelesin, cedefingol, celecoxib, chlorambucil, cirolemycin, cisplatin, cladribine, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, droloxifene, dromostanolone, duazomycin, edatrexate, eflomithine, elsamitrucin, enloplatin, enpromate, epipropidine, epirubicin, erbulozole, esorubicin, estramustine, estramustine, etanidazole, etoposide, etoposide, etoprine, fadrozole, fazarabine, fenretinide, floxuridine, fludarabine, fluorouracil, flurocitabine, fosquidone, fostriecin, fulvestrant, gemcitabine, gemcitabine, hydroxyurea, idarubicin, ifosfamide, ilmofosine, interleukin II (IL-2, including recombinant interleukin II or rIL2), interferon alpha-2a, interferon alpha-2b, interferon alpha-n1, interferon alpha-n3, interferon beta-1a, interferon gamma-I b, iproplatin, irinotecan, lanreotide, letrozole, leuprolide, liarozole, lometrexol, lomustine, losoxantrone, masoprocol, maytansine, mechlorethamine hydrochlride, megestrol, melengestrol acetate, melphalan, menogaril, mercaptopurine, methotrexate, methotrexate, metoprine, meturedepa, mitindomide, mitocarcin, mitocromin, mitogillin, mitomalcin, mitomycin, nitosper, mitotane, mitoxantrone, mycophenolic acid, nelarabine, nocodazole, nogalamycin, ormnaplatin, oxisuran, paclitaxel, pegaspargase, peliomycin, pentamustine, peplomycin, perfosfamide, pipobroman, piposulfan, piroxantrone hydrochloride, plicamycin, plomestane, porfimer, porfiromycin, prednimustine, procarbazine, puromycin, puromycin, pyrazofurin, riboprine, rogletimide, safingol, safingol, semustine, simtrazene, sparfosate, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, triethylenemelamine, trimetrexate, triptorelin, tubulozole, uracil mustard, uredepa, vapreotide, verteporlin, vinblastine, vincristine sulfate, vindesine, vinepidine, vinglycinate, vinleurosine, vinorelbine, vinrosidine, vinzolidine, vorozole, zeniplatin, zinostatin, zoledronate, and zorubicin. These and other antineoplastic therapeutic agents are described, for example, in Goodman & Gilman's The Pharmacological Basis of Therapeutics, McGraw-Hill Professional, 10th ed., 2001.

In various aspects, the therapeutic compound is an anti-inflammatory selected from the group consisting of glucocorticoids; kallikrein inhibitors; corticosteroids (e.g. without limitation, prednisone, methylprednisolone, dexamethasone, or triamcinalone acetinide); anti-inflammatory agents (such as without limitation noncorticosteroid anti-inflammatory compounds (e.g., without limitation ibuprofen or flubiproben)); vitamins and minerals (e.g., without limitation zinc); anti-oxidants (e.g., without limitation carotenoids (such as without limitation a xanthophyll carotenoid like zeaxanthin or lutein)) and agents that inhibit tumor necrosis factor (TNF) activity, such as without limitation adalimumab (HUMIRA®), infliximab REMICADE®), certolizumab (CIMZIA®), golimumab (SIMPONI®), and etanercept (ENBREL®).

In various aspects, the therapeutic compound isM-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNF\ddot{γ}, TNF1, TNF2, G-CSF, Meg-CSF, GM-CSF, thrombopoietin, stem cell factor, and erythropoietin. Additional growth factors for use herein include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor \ddot{γ}, cytokine-induced eutrophils chemotactic factor 1, cytokine-induced eutrophils, chemotactic factor 2 \ddot{γ}, cytokine-induced eutrophils chemotactic factor 2 \ddot{γ}, \ddot{γ} endothelial cell growth factor, endothelin 1, epithelial-derived eutrophils attractant, glial cell line-derived neutrophic factor receptor \ddot{γ} 1, glial cell line-derived neutrophic factor receptor \ddot{γ} 2, growth related protein, growth related protein \ddot{γ}, growth related protein \ddot{γ}, growth related protein \ddot{γ}, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor \ddot{γ}, nerve growth factor nerve growth factor receptor, neurotrophin-3, neurotrophin-4, pre-B cell growth stimulating factor, stem cell factor, stem cell factor receptor, transforming growth factor \ddot{γ}, transforming growth factor \ddot{γ}, transforming growth factor \ddot{γ}, transforming growth factor \ddot{γ}.2, transforming growth factor \ddot{γ}, transforming growth factor \ddot{γ}, transforming growth factor \ddot{γ}, latent transforming growth factor \ddot{γ}, transforming growth factor \ddot{γ} binding protein I, transforming growth factor \ddot{γ} binding protein II, transforming growth factor \ddot{γ} binding protein III, tumor necrosis factor receptor type I, tumor necrosis factor receptor type II, urokinase-type plasminogen activator receptor, intracellular sigma peptide (ISP), and chimeric proteins and biologically or immunologically active fragments thereof.

Method are also provided for with anticoagulation drugs. Including, for example and without limitation, plavix, aspirin, warfarin, heparin, ticlopidine, enoxaparin, Coumadin, dicumarol, acenocoumarol, citric acid, lepirudin and combinations thereof..

Methods in this aspects overcome the effects of these anticoagulant drugs which would be extremely helpful in surgery.

### II. Pharmaceutical Composition

The disclosure provides a pharmaceutical composition comprising a nanoparticle of the disclosure. In various aspects, the pharmaceutical composition is a unit dose formulation. In various aspects, the pharmaceutical composition is an intravenous administration formulation. In various aspects, the pharmaceutical composition is lyophilized or a powder. In various aspects the pharmaceutical composition further comprises polyacrylic acid.

In various aspects, a topical formulation is provided. Internal and external uses are provided wherein. The pharmaceutical composition for topical administration optionally includes a carrier, and is formulated as a solution, emulsion, ointment or gel base. The base, for example, optionally comprises one or more of the following: petrolatum, lanolin, polyethylene glycols, beeswax, mineral oil, diluents such as water and alcohol, and emulsifiers and stabilizers. Thickening agents are optionally present in a pharmaceutical composition for topical administration. In certain aspects, a solvent is in the formulation, the solvent including for example and without limitation, MMP, DMSO or a similar compound.

The disclosure provides pharmaceutical compositions formulated for delivery of nanoparticles at 1 mg/kg to 1 g/kg, 10 mg/kg to 1 g/kg, 20 mg/kg to 1 g/kg, 30 mg/kg to 1 g/kg, 40 mg/kg to 1 g/kg, 50 mg/kg to 1 g/kg, 60 mg/kg to 1 g/kg, 70 mg/kg to 1 g/kg, 80 mg/kg to 1 g/kg, 90 mg/kg to 1 g/kg, 10 mg/kg to 900 mg/kg, 10 mg/kg to 800 m/kg, 10 mg/kg to 700 mg/kg, 10 mg/kg to 600 mg/kg, 10 mg/kg to 500 mg/kg, 10 mg/kg to 400 mg/kg, 10 mg/kg to 300 mg/kg, 10 mg/kg to 200 mg/kg, 10 mg/kg to 100 mg/kg, 10 mg/kg to 75 mg/kg, 10 mg/kg to 50 mg/kg, 50 mg/kg to 900 mg/kg, 100 mg/kg to 800 mg/kg, 200 mg/kg to 700 mg/kg, 300 mg/kg to 600 mg/kg, 400 mg/kg to 500 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg, 300 mg/kg, 400 mg/kg, 500 mg/kg, 600 mg/kg, 700 mg/kg, 800 mg/kg, 900mg/kg, 1000 mg/kg, or more.

Single dose administrations are provided, as well as multiple dose administrations. Multiple dose administration includes those wherein a second dose is administered within minutes, hours, day, weeks, or months after an initial administration. In methods that

### III. Use

A method of treating an condition in an individual is provided comprising the step of administering the nanoparticle of the disclosure to a patient in need thereof in an amount effective to treat the condition. In various aspects, the individual has a bleeding disorder. Methods are provided wherein the nanoparticle is administered in an amount effective to reduce bleeding time by more than 15%, by more than 20%, by more than 25%, or by more than 30% compared to no administration or administration of saline. In various aspects, the method is used wherein the bleeding disorder is a symptom of a clotting disorder, an acquired platelet function defect, a congenital platelet function defect, a congenital protein C or S deficiency, disseminated intravascular coagulation (DIC), Factor II deficiency, Factor V deficiency, Factor VII deficiency, Factor X deficiency, Factor XII deficiency, Hemophilia A, Hemophilia B, Idiopathic thrombocytopenic purpura (ITP), von Willebrand's disease (types I, II, and III), megakaryocyte/platelet deficiency. In various aspects, a method is provided wherein the condition is thrombocytopenia arising from chemotherapy and other therapy with a variety of drugs, radiation therapy, surgery, accidental blood loss, and other specific disease conditions. In various aspects, a method is provided wherein the condition is aplastic anemia, idiopathic or immune thrombocytopenia (ITP), including idiopathic thrombocytopenic purpura associated with breast cancer metastatic tumors which result in thrombocytopenia, systemic lupus erythematosus, including neonatal lupus syndrome, metastatic tumors which result in thrombocytopenia, splenomegaly, Fanconi's syndrome, vitamin B12 deficiency, folic acid deficiency, May-Hegglin anomaly, Wiskott-Aldrich syndrome, paroxysmal nocturnal hemoglobinuria, HIV associated ITP and HIV-related thrombotic thrombocytopenic purpura; chronic liver disease; myelodysplastic syndrome associated with thrombocytopenia; paroxysmal nocturnal hemoglobinuria, acute profound thrombocytopenia following C7E3 Fab (Abciximab) therapy; alloimmune thrombocytopenia, including maternal alloimmune thrombocytopenia; thrombocytopenia associated with antiphospholipid antibodies and thrombosis; autoimmune thrombocytopenia; drug-induced immune thrombocytopenia, including carboplatin-induced thrombocytopenia, heparin-induced thrombocytopenia; fetal thrombocytopenia; gestational thrombocytopenia; Hughes' syndrome; lupoid thrombocytopenia; accidental and/or massive blood loss; myeloproliferative disorders; thrombocytopenia in patients with malignancies; thrombotic thrombocytopenia purpura, including thrombotic microangiopathy manifesting as thrombotic thrombocytopenic purpura/hemolytic uremic syndrome in cancer patients; autoimmune hemolytic anemia; occult jejunal diverticulum perforation; pure red cell aplasia; autoimmune thrombocytopenia; nephropathia epidemica; rifampicin-associated acute renal failure; Paris-Trousseau thrombocytopenia; neonatal alloimmune thrombocytopenia; paroxysmal nocturnal hemoglobinuria; hematologic changes in stomach cancer; hemolytic uremic syndromes in childhood; and hematologic manifestations related to viral infection including hepatitis A virus and CMV-associated thrombocytopenia. In various aspects, a method is provided wherein the condition arises from treatment for AIDS which result in thrombocytopenia. In various aspects, the treatment for AIDS is administration of AZT.

In various aspect, the individual being treated is suffering from a wound healing disorders, trauma, blast trauma, a spinal cord injury, hemorrhagic stroke, hemorrhaging following administration of TPA, or intraventricular hemorrhaging which is seen in many conditions but especially acute in premature births.

### EXAMPLE 1

The first model for testing nanoparticles for control of bleeding was the hamster cremaster prep in which the microvessels were exposed and injured by administering fluorescein and exciting it with a UV light to damage the microvessels and induce activation of platelets. Time to form a clot was recorded.

The first nanoparticle was a 4-arm PEG with a molecular weight of 10,000 g/mol. The PEG molecule was activated with N,N′-Carbonyldiimidazole (CDI) and coupled RGD to the ends. It was thought this nanoparticle would act as a bridge between activated platelets and decrease the clot formation time, but what was found was that it exacerbated bleeding dramatically.

Based on this results a larger molecular weight PEG was proposed to bridge between the particles, but as PEG gets larger, it takes on conformations that do not favor exposing the peptide. Thus a core-based system was designed to promote the presentation of the peptide and be large enough to bridge between activated platelets to participate in clot formation.

### EXAMPLE 2

The degradation rate of the nanoparticles is modulated via the molecular weight and ratio of lactic acid to glycolic acid units. One of the major attractions of using PLGA beyond its use in FDA approved products is that it can be used it to deliver drugs, leveraging drug delivery technology on the synthetic platelet platform. The PLL provides free amines onto which the PEG can be coupled using traditional coupling chemistry based on N,N′-Carbonyldiimidazole (CDI). One attraction of PEG being attached to PLGA-b-PLL is that multiple PEG arms can be attached. The multiple branches increase the propensity for surface segregation and lead to greater exposure of the functional moiety. The PEG makes the nanoparticles hydrophilic allowing them to travel through the bloodstream and reducing the propensity for the nanoparticles to collect in the liver. PEG is a non-toxic, non-thrombogenic material, and it allows the nanoparticles to bond specifically with their targets. The RGD moiety, or a variation on it, provides functionality to bind with activated platelets and augment their clotting behavior. Chemical modification with the RGD peptide or one of its variants (RGDS, GRGDS) has been shown to augment platelet behavior in other systems. The RGD moiety is seen in many systems; in platelets it appears when the platelets are activated, releasing fibrinogen which causes aggregation of the platelets at the injury site.

A variety of tools were used to characterize the nanoparticles including 1H-NMR, UV-vis, amino acid analysis, and dynamic light scattering. From this analysis, it was shown that the core of the nanoparticles is approximately 170 nm, and that the length of the PEG arms varied from 90 to 150 nm by varying the PEG molecular weight from 1500 Da to 8000 Da. Three variants on the RGD moiety (RGD, RGDS, and GRGDS) were used and the coupling efficiency was approximately 35% for all of the peptides.

### EXAMPLE 3

An in vitro system was developed for high throughput screening of the coagulation efficiency of the synthetic platelets with the platelets labeled using CellTracker green following to facilitate ease of analysis. Essentially, this assay involves activating platelets which have been previously labeled with CellTracker and looking at the number that bind to surfaces modified with a polymer systems under agitation. This assay was validated with collagen. This system allowed one to efficiently and independently vary the PEG molecular weight and RGD motif (i.e. RGD, RGDS, and GRGDS).

In preliminary work, activated nanoparticle binding was augmented with PEG 4600 and the GRGDS peptide led to efficient adhesion and aggregation. It has been established that by introducing flanking amino acids to the RGD motif, an active conformation is obtained. This bioactivity in turn influences integrin affinity for the RGD moiety, and increases cellular attachment. The GRGDS peptide was shown to provide good binding and adhesion of the activated platelets.

Previous work by others has shown that the length of the peptide can effect its temperature stability as well as production costs. These synthetic platelets were designed to be stable at room temperature to facilitate their administration in the field.

### EXAMPLE 4

Following optimization in vitro, a test of the efficacy of nanoparticles was performed in a femoral artery partial severance model. Approximately 20 mg/ml of particles was injected intravenously and imaged the blood flow to determine the clotting time.

Systemic administration of the functionalized nanoparticles with PEG 4600 and the GRGDS peptide halved clotting time in the femoral artery. Scanning electron microscopy of the excised clot showed synthetic platelets (marked by the red arrow) intimately associated with the clot. Importantly, no adverse effects including stroke or sings of breathing problems associated with particle build up or thrombosis in the CNS or lungs were seen. Biodistribution data indicated that unbound synthetic platelets cleared within 24 hours, and no differences were seen with or without the injury. These data demonstrate the synthetic platelets actively augment clotting and are an important tool in studying the role of hemostasis following CNS trauma.

### EXAMPLE 5

The first observed phenomena following mechanical trauma to the CNS is the rupture of microvessels. This phenomenon is followed by an injury cascade that includes ischemia, anoxia, free-radical formation, and excitotoxicity that occur over hours and days following injury. If one can halt the initial hemorrhaging, the question arose as to whether can one inhibit the secondary degeneration and preserve tissue and function.

The extent of hemorrhaging has been correlated with the degree of functional deficits following CNS trauma in humans. It is also correlated with the extent of injury in rodent models. While there is limited literature looking at halting hemorrhaging since the current drugs to induce hemostasis have risks for causing strokes following CNS trauma, early clinical evidence suggests that inducing hemostasis by administering rFVIIa, does improve outcomes. This result suggests that a means to halt bleeding that is more effective than rFVIIa has the potential to significantly improve outcomes.

Ultimately, the nanoparticles are bound into the clot at the injury site. For CNS injury, this result means a platform is provided for localized, targeted drug delivery to provide neuroprotection.

There are a number of factors that can be incorporated into the nanoparticles. Using techniques similar to fabrication of the nanoparticle cores, PLGA-based nanoparticles were prepared with diameters on the order of the synthetic platelet cores that delivery ciliary neurotrophic factor (CNTF), which has been shown by others to be neuroprotective in a number of CNS injuries and diseases. These nanoparticles delivery nanogram quantities of CNTF for 14 days and the growth factor is bioactive. Nanoparticles loaded with CNTF show delivery over 20 days.

Results also by others demonstrated delivery of glial cell line-derived neurotrophic factor (GDNF) from PLGA particles in a number of injury models, as well as delivery of triamcinolone, a steroid, which has been implicated in reducing inflammation, aiding in reducing vessel leakiness and providing protection following CNS injury.

### EXAMPLE 6

Nanoparticle consisting of poly(lactic-co-glycolic acid)-poly-L-lysine (PLGA-PLL) block copolymer cores were conjugated to polyethylene glycol (PEG) arms terminated with RGD functionalities. Conjugation of PEG to PLGA-PLL was confirmed using 1-NMR. Nanoparticles were fabricated using a single emulsion solvent evaporation technique, and the size was confirmed by scanning electron microscopy (SEM). The subsequent conjugation of GRGDS to PLGA-PLL-PEG nanoparticles was quantified using amino acid (AA) analysis. Dynamic light scattering was used to determine the hydrodynamic volume of the spheres.

### EXAMPLE 7

The nanoparticles were tracked by loading the nanoparticle cores with Coumarin 6 (C6) which can be detected using excitation and emission wavelength pairs of 444/538 nm via HPLC. This allows one to quantify the biodistribution of the nanoparticles. C6 does not alter the size or behavior of the particles, and because the C6 is so hydrophobic, 99% remains in the PLGA cores for 7 days.

### EXAMPLE 8

The disclosure provides applications of the nanoparticles for trauma in the CNS. Based on preliminary evidence, the nanoparticles accumulate in the clot. In the case of CNS trauma, this means that the particles will be in the CNS at the area where the blood-brain barrier (BBB) has been compromised.

Triamcinolone has the capacity to help control inflammation and seal vessels as well as protect neural tissue. Furthermore, it has been delivered PLGA particles. Triamcinolone acetate is therefore encapsulated using the single emulsion process and quantify release using HPLC.

### EXAMPLE 9

In preliminary work, a femoral artery injury model was used. It is a very clean model that allows simple assessment of the impact of a therapy on bleeding. To determine the efficacy of the nanoparticles in a blunt trauma model as well as to gain critical data regarding the mechanism and impact of the nanoparticles on clotting, a liver injury model coupled is used with assessments of coagulation over time.

Male Sprague-Dawley rats were anesthetized with isoflurane. The animal's temperature was maintained using a heating pad and monitored throughout the experiment using a temperature probe. An arterial catheter was used for measuring blood pressure and blood draws, and a venous catheter was used for administration of the agent being tested. The abdominal cavity was opened, and the median lobe of the liver is cut sharply 1.3 cm from the superior vena cava following. The cavity was immediately closed, and the experimental agent was delivered.

Blood samples were drawn immediately before the injury, at 5 minutes post injury, and at 30 minutes post injury. Animals were maintained for 60 minutes or until death. At the end of 60 minutes, pre-weighed sponges were used to collect the blood in the abdominal cavity to determine blood loss. All the major organs were collected for histology and biodistribution of the nanoparticles.

The data from this work provides critical information into the efficacy, safety, and mechanism of the nanoparticles. If the nanoparticles do not show significantly augmented hemostasis, the terminal peptide is altered to augment binding to activated platelets.

### EXAMPLE 10

A controlled cortical impact (CCI) model in male Sprague-Dawley rats for the TBI work was used. The CCI model combines physiologically relevant pathological and behavioral outcomes with a highly quantifiable system. A severe model was used with a Pittsburg precision impactor device with an impact depth of 2 mm following.

This injury leads to significant motor and cognitive deficits that was quantified using a rotorod test and Morris Water Maze test (MWM) and correlated with histological outcomes including lesion size, gliosis, and amount of positive neural tissue.

This study determined how effective the nanoparticles were at halting hemorrhaging following TBI, and how the induction of hemostasis impact recovery.

This approach also provided a simple route of administration for locally delivered steroids, namely intravenous administration. Current approaches to deliver these factors focus on implantable pumps and catheters because the factors cannot cross the blood-brain barrier, have short half-lives, and can cause side effects. However, implantable catheters in the CNS carry risks, especially for patients compromised by trauma.

Having a simply administered but effectively targeted system mitigates a number of the delivery issues associated with neurotrophic administration.

### EXAMPLE 11

Nanoparticles were synthesized from poly (lactic-co-glycolic acid)-poly-_{L}-lysine (PLGA-PLL) block copolymer conjugated with polyethylene glycol (PEG) arms [1]. Spherical nanoparticles were fabricated using a nano precipitation method as described herein. Dexamethasone was dissolved in a solvent, and the appropriate amount of polymer was also dissolved and mixed with the drug. The drug/polymer solution was pipetted dropwise into spinning 1× PBS. The resultant solution was allowed to stir uncovered for approximately 20 min at room temperature. After the nanospheres stir hardened, the pH was adjusted down to 3.0-2.7 to induce flocculation. This pH range was found to be useful for flocculation to occur. The nanospheres were purified by centrifugation (500 g, 3 min, 3×), resuspended in deionized water, frozen, and freeze-dried on a lyophilizer. A release study was performed by dissolving 10 mg of nanospheres into 1 mL 1× PBS, repeated in triplicate.

Size of the nanospheres was determined by dynamic light scattering (DLS). Conformation of size and morphology was determined by a scanning electron microscope (SEM). The amount of drug was determined by dissolving spheres in DMSO and running on a UV-Vis. Release study data was gathered at various time points and was run on UV-Vis to determine how dexamethasone elutes out of the nanoparticles over time.

### EXAMPLE 12

The yield and time to make product has been significantly reduced by determining the shortest times necessary for intermediate treatment steps. Yield is significantly increased using centrifugation to collect PLGA-PLL-PEG after precipitating. Yield is also significantly increased with nanoprecipitation nanoparticle formation method and even further increased if using the poly(acrylic acid) coacervate precipitation technique for nanoparticle collection.

Once the PLGA-PLL-PEG is synthesized, the active peptide such as GRGDS needs to be coupled to the polymer.

When the quad block polymer (PLGA-PLL-PEG-peptide) was used, yield of spheres was extremely low. Since the peptide was the most expensive portion of the polymer, a method was employed to form spheres from the triblock (PLGA-PLL-PEG) and then attach the peptide to the spheres themselves.

Conjugation of the peptide to triblock nanoparticles led to approx. 50% conjugation efficiency (calculated as the arginine to lysine ratio).

However, it was found that an extra rinse step of the nanospheres before amino acid analysis led to significant loss of the peptide with a conjugation efficiency of 11%. Upon scaling the reaction up for a 1 g batch of nanospheres, the conjugation efficiency essentially dropped to 0%. Therefore, a method was pursued that would allow one to make the entire quad block polymer and with at least comparable yield produce nanoparticles with a tight size distribution.

This approach led to the manufacture of a quadblock polymer prior to the formation of the nanoparticle. The quadblock conjugation efficiency was approximately 80%, but dropped to 13% after nanosphere formation using the nanoprecipitaiton technique with and without poly(acrylic acid). Finally, the quadblock was made by reactivating the polymer with CDI in DMSO immediately prior to the addition of the peptide. This step increases the conjugation of peptide to above 50% (n=3).

Emulsion Method

The emulsion method succeeds in making spheres of diameter between 326-361 nm.

The emulsion method stir-hardens the nanospheres in 50 ml of 5% PVA in deionized water. Scaling up the production of nanospheres using this method requires large volumes of solution for stir hardening. This observation, coupled with the fact that prior methods added the peptide for the conjugation step after forming the particles, means that a very large amount of peptide would be needed for the large volume of solution to achieve a reasonable coupling efficiency.

For the nanoprecipitation method, scaled down version, stir hardening in 10 ml PBS was carried out with simultaneous conjugation of the peptide. This step adds a sufficient amount of peptide. The nanoprecipitation method also lends itself to the formation of nanoparticles with the quadblock polymer eliminating the need for a post-fabrication coupling reaction.

There are a number of fundamental issues identified with nanoparticles, including uniformity of particles, aggregation of particles, challenges in resuspending nanoparticles and challenges of resuspending following lyophilization

Groups have come up with a number of approaches to deal with these challenges. For example, one can have a lyoprotectant to resuspend small nanoparticles following lyophilization. (Sauaia et al., J Trauma 38, 185 (1995)), Champion, et al., J Trauma 54, S13 (2003)). Other found that through nanoprecipitation technique coupled with the use of poly(acrylic acid) to flocculate the particles, the need to add a lyoprotectant to the solution was avoided.

Nanoprecipitation

The nanoprecipitation method uses dropwise addition of polymer dissolved in a water miscible solvent such as acetonitrile to make spheres of consistent size (Regel, et al., Acta Anaesthesiol Scand Suppl 110, 71 (1997); Lee, et al., Expert Opin Investig Drugs 9, 457 (2000); Blajchman, Nat Med 5, 17 (1999); Lee, et al., Br J Haematol 114, 496 (2001)).

Poly(Acrylic Acid) Coacervate Precipitation

This method modified from (Regel, et al. (1997); Kim, et al., Artif Cells Blood Substit Immobil Biotechnol 34, 537 (2006)) was employed to increase yield of nanoparticles and to reduce aggregation of spheres during centrifugation and lyophilization steps as had previously been observed. The precipitation allows for gentle centrifugation <500 g.

The size reproducibility has thus far been shown to be an advantage over the emulsion and nanoprecipitation alone methods which is highly dependent on sonication conditions to make a homogenous size distribution. SEM image shows morphology of nanoparticles and homogeneity of size. Histogram inlay was made from 100 measurements of nanoparticle diameter, and shows size distribution is centered around 236.1 nm +/−56.6 nm.

Method for Making PAA-Coated Nanoprecipitated Synthetic Platelets

PLGA (Resomer 503H) was purchased from Evonik Industries. Poly-1-lysine and PEG (˜4600 Da MW) were purchased from Sigma Aldrich. All reagents were ACS grade and were purchased from Fisher Scientific. PLGA-PLL-PEG coblock polymer was made using standard bioconjugation techniques as previously described (Lavik et al).

Quadblock Conjugation

PLGA-PLL-PEG was dissolved in anhydrous DMSO to a concentration of 100 mg/ml. Two molar equivalents of CDI were added to reactivate the PEG groups and stirred for 1 hour. Twenty five mg of oligopeptides (GRGDS or GRADSP) was dissolved in 1 ml DMSO and added to the stirring polymer solution. This mixture was reacted for 3 hours, and then transferred to dialysis tubing (SpectraPor 2 kDa MWCO). Dialysis water was changed every half hour for 4 hours with Type I D.I. water. The product was then snap-frozen in liquid nitrogen and lyophilized for 2 days.

Nanoprecipitation

The resulting quadblock copolymer PLGA-PLL-PEG-GRGDS was then dissolved to a concentration of 20 mg/ml in acetonitrile. This solution was added dropwise to a stirring volume of PBS. The general rule is to use twice the volume of PBS as acetonitrile. Precipitated nanoparticles formed as the water-miscible solvent dissipates. However, to scale up to quantities greater than 300 mg starting quadblock, it was found that priming the precipitation volume with acetonitrile reduced the spontaneous formation of aggregates. Solvent:water ratios were adjusted throughout the precipitation process so that the final concentration in the precipitation volume is 2:1 PBS:acetonitrile. The particles were then stir-hardened for 3 hours. Particles were then collected using centrifugation @ 15000 g and rinsing with PBS 3 times. Alternatively, particles were collected using the coacervate precipitation method.

Coacervate Precipitation

One mass equivalent of dry poly(acrylic acid) was added to the stirring particle suspension. 1% w/v pAA was then added to the stirring suspension until flocculation occurs. Stirring was paused momentarily after each addition of pAA to observe flocculation. After 5 minutes, the flocculated particles were collected by centrifugation at 500 g, and rinsed 3 times with 1% pAA (centrifuging @ 500 g, 2 m, 4C between rinses). On the final rinse, particles were resuspended with D.I. water, snap-frozen and lyophilized for 2-5 days, depending on the final volume of water.

Resuspension

Particles were massed and resuspended to a concentration of 20 mg/ml in 1×PBS. Particles are either vortexed to resuspend, or alternatively vortexed and briefly sonicated at 4W to a total energy of 50 J using a probe sonicator (VCX-130, Sonics & Materials, Inc.).

### EXAMPLE 13

Explosions cause of the majority of injuries in the current conflicts accounting for 79% of combat related injuries. Uncontrolled bleeding is the leading cause of death in battlefield traumas. Following injury, hemostasis is established through a series of coagulatory events including platelet activation. However, with severe injuries, these processes are insufficient and result in uncontrolled bleeding. Immediate intervention is one of the most effective means of minimizing mortality associated with severe traumas, and yet the only available treatments in the field are pressure dressings and absorbent materials which are effective for exposed wounds, but cannot be used for internal injuries. A therapy is needed that can be administered in the field by a medic to complement the pressure dressings and stop bleeding.

Nanoparticles described herein halve bleeding time in a femoral artery injury model as discussed above. These nanoparticles act essentially as synthetic platelets and are stable at room temperature, and can be administered intravenously. Because they can stop bleeding, are used in a model of blast trauma to determine whether they can improve survival after explosions as well as preserve tissue leading to better functional outcomes.

Preparation of Nanoparticles

Poly(lactic-co-glycolic acid)-based nanoparticles with poly(ethylene glycol) (PEG) arms and the RGD peptide to target activated platelets were fabricated. PLGA-PLL-PEG-GRGDS for the synthetic platelets or PLGA-PLL-PEG-GRADSP was synthesized using protocols described previously. The polymer was dissolved at a concentration of 20 mg/ml in acetonitrile containing coumarin-6 (C6), a fluorescent dye used to track the particles after injection (loaded at 1% w/w). This solution was added drop wise to a volume of stirring PBS, twice that of the acetonitrile. Precipitated nanoparticles form as the water-miscible solvent is displaced. The particles were then stir-hardened for 3 hours. One mass equivalent of dry poly(acrylic acid) (pAA) (Sigma, MW=1,800) is added to the stirring particle suspension. 1% w/v pAA is then added to the stirring suspension until flocculation occurs, approximately 10 ml. After 5 minutes, the flocculated particles are collected by centrifugation at 500 g, and rinsed 3 times with 1% pAA (centrifuging at 250 g, 2 min, 4 deg C. between rinses). On the final rinse, particles are resuspended to approximately 10 mg/ml with deionized water, snap-frozen in liquid nitrogen and lyophilized for 3 days. Particles were collected using the coacervate precipitation method described below.

The particles were characterized in vitro using ROTEM analysis and in vivo in a mouse model of full body blast trauma at 20 psi. Coagulation assays, using Sprague Dawley rat blood, were performed using the ROTEM's NATEM test in the presence of either saline, GRGDS conjugated synthetic platelets, or the Nanoparticle control, GRADSP conjugated nanoparticles. The blood collection method (cardiac puncture) is rigidly followed to minimize variability in the highly sensitive NATEM test. All animal procedures were approved and undertaken according to the guidelines set by Case Western Reserve University's institutional animal care and use committee.

In a second appraoch to preparing the nanoparticles, the the polymer (PLGA-PLL-PEG-GRGDS) is first made and and then formed into nanospheres.

Animal Model

A blast trauma injury model was generated as follows. A custom-built shock tube located was used to induce blast overpressure. Mylar sheets are placed between the compression chamber and the tube to attain peak pressures. During blast exposure, the pressure versus time profile will be measured using a piezoelectric sensor (model 137A22 Free-Field ICP Blast Pressure Senor, PCB Piezotronics) placed axial to the blast pressure source. One sensor (model 1022A06 ICP Dynamic Pressure Sensor, PCB Piezotronics) is installed in a threaded intra-tube canal located perpendicular to the induced pressure wave will also measure the induced pressure time profile. A portable analog to the digital data acquisition system (Model DASH 8HF, Astro-Med Inc.) collects the data from all pressure transducers at 250 kHz per channel.

Prior to blast exposure, two mice were anesthetized with a ketamine/xylazine solution. While under anesthesia the mice were weighted, then the hind right leg was shaved using an electric razor followed by a straight edge razor in order to collect physiological response to blast. The anesthetized animals were placed on a heating pad. A thigh clip sensor was placed on the shaved hind leg which is connected to the MouseOx physiological monitoring system. The mice were monitored for 20 minutes post-injection of anesthetics, and then were placed in a custom built restraint harness (FIG. 1) and exposed to a whole body blast.

After the blast exposure, animals were removed, returned to the warm pad, and the thigh clip was reapplied for monitoring during the for a one-hour evaluation period. The MouseOx system was used to collect the several physiological parameters such as heart rate, breath rate, oxygen saturation, pulse distention and breath distention. Within 10 minutes of the blast, the treatment (Synthetic platelets, 50 ul of a 20 mg/ml solution in Lactated Ringers; Nanoparticle control, GRADSP-particles, 50 ul of a 20 mg/ml solution in Lactated Ringers; NovoSeven, 50 ul; Lactated Ringers, 50 ul; or no treatment) was administered intravenously via the tail vein.

If the animal died before the one-hour assessment, the tissues were quickly collected for histological analysis. If the animals survived the one-hour time assessment, they were overdosed with ketamine/xylazine and perfused with 4% paraformaldehyde as described below, and tissues were then collected for histological assessment. A small cohort of animals was allowed to survive for up to 3 weeks post injury to determine if the survival in the acute phase correlated with long term survival and to see if there were complications associated with the administration of the synthetic platelets or nanoparticle controls.

The person performing the blast trauma and the person administering the treatment were blinded to the treatments, and death was independently recorded by a person also blinded to the treatment. The no injection group (n=3) is included as a reference, but is not included in the statistics. Survival was analyzed with a binomial logistic regression with chi-squared tests between odds-ratios (SAS).

Before the synthetic platelets or controls could be administered, the blast model in mice had to be validated. The lethality study began by exposing animals to a 15 PSI blast exposure. All mice from this group survived the one-hour assessment. As such, the overpressure was increased and a second group of mice was exposed to a pressure of 20 PSI. At this level, a 40% lethality rate was determined. A third group of animals were exposed to an overpressure of 25 PSI and we found that 90% of the animals died within the first hour following blast exposure.

The physiological parameters showed consistency with respect to the animals exposed to the higher pressure exhibited a decrease in health status. Mice exposed to 25 PSI overpressure were found to have the lowest level of oxygen saturation as compared to all other groups.

The extent of lung injury was quantified by using eosin-only stained sections of the lungs. Images were taken of three regions of interest (ROI) in each lung tissue section. Eosin is a negatively-charged molecule that stains positively charged tissue. In particular, it stains red blood cells a distinctive bright red color that allows them to be easily distinguished from the surrounding tissue and provides a simple means to characterize the degree of hemorrhaging in the lungs.

These three eosin images were converted to black and white and optical density readings are collected in order to determine the level of hemorrhaging in the lung tissue. After the percent-injured area was calculated, significance was determined at and was reported as mean ±SEM. In particular, there is a significant increase in lung injury at 20 psi. This observation correlates well with the physiological findings as well as the lethality of the blast model, and based on this, we determined that an overpressure of 20 psi would be appropriate for testing the impact of the synthetic platelets on survival following blast injury.

Analysis

One-hour post exposure to 20 psi, surviving animals were sacrificed by transcardially perfused with saline (0.9% sodium chloride) followed by fixative solution containing 4% formaldehyde. All major organs (lungs, brain, kidney, liver, GI) were collected and stored in a fixative solution containing 15% sucrose. After 48 hours, the lungs were placed in OCT embedding medium and allowed to freeze on dry ice. The samples were then cut and stained with hematoxylin and eosin (H&E) and ‘eosin only’. Eosin only sections were used to quantify lung injury. Images were taken of three regions of interest (ROI) in each lung tissue section. Using Image J software (NIH), the images were converted to grey scale and optical density readings were collected in order to determine the level of hemorrhaging in the lung tissue. FIG. 1 demonstrates one example of how each section was analyzed. After the percent injured area was calculated, significance was determined at and was reported as mean ±SD. Histological statistical analysis was calculated with a two way ANOVA followed by a post hoc LSD test with significance achieved with p<0.05.

Liver, kidneys, spleen, lungs, and brain were harvested and lyophilized for the biodistribution assay. The dry weight of the whole organ was recorded and 100-200 mg of dry tissue was homogenized (Precellys 24) and incubated overnight in acetonitrile at 37 C. This dissolved any synthetic platelets present in the tissue and left the C6 in the organic solvent solution. Tubes were then centrifuged at 15,000 g for 10 minutes to remove solid matter and supernatant was tested on the HPLC. Mobile phase was 80% acetonitrile, and 20% aqueous (8% acetic acid). Stationary phase was a Waters Symmetry C18 Column, 100 Å, 5 μm, 3.9 mm×150 mm. Samples that oversaturated on the fluorescence detector (450/490 nm ex/em) were diluted and re-run. Based on the known C6 loading and injection volume of particles, data is represented as % of particles injected.

Coagulation assays, using Sprague Dawley rat blood, were performed using the ROTEM's NATEM test in the presence of either saline, GRGDS conjugated synthetic platelets, or the Nanoparticle control, GRADSP conjugated nanoparticles. The blood collection method (cardiac puncture) is rigidly followed to minimize variability in the highly sensitive NATEM test. All animal procedures were approved and undertaken according to the guidelines set by Case Western Reserve University's institutional animal care and use committee.

A 5 ml syringe was loaded with 0.5 ml of 3.8% disodium citrate prepared in 1× PBS. Rats were anesthetized with a ketamine:xylazine rodent cocktail (90:10 mg/kg, i.p.), and heartbeat palpated. The needle was then slowly advanced while aspirating until a flash occurs. 4.5 ml of blood was collected to mix with the anticoagulant solution at a 1:9 ratio (solution:blood). For a given run, the cup of blood consisted of: 300 μl citrated blood, 20 μl starTEM reagent (0.2 mM calcium chloride), 20 μl synthetic platelets (1.25 or 2.5 mg/ml), totaling a 340 μl sample. To account for time dependency on coagulation tests, the experimental design was created such that a block of 4 NATEM tests were run simultaneously on a single ˜1.2 cc aliquot of blood, where saline was always included as one of the four tests to allow for direct comparison. The main outcomes analyzed were clotting time, clot formation time and maximum clot firmness as defined by ROTEM. The raw data was analyzed using a generalized linear model, with run time as blocks and with Tukey comparisons between groups. The main outcomes considered include the standard ROTEM parameters clotting time (CT), clot formation time (CFT), the sum of the two (CT+CFT), and maximum clot firmness (MCF). CT is defined as the time from the start of the assay until the initial clotting is detected (thickness=2 mm). CFT is defined as the time between the initial clot (thickness=2 mm) until a clot thickness of 20 mm is detected. MCF is defined as the maximum thickness (in mm) that a clot reaches during the duration of the test.

Results

In these results of the 21 animals exposed to the 20 psi blast and administered synthetic platelets, only one animal died prior to the one hour time point. This result is significantly better than the no injection control group. Survival was analyzed with a binomial logistic regression with chi-squared tests between odds-ratios (SAS). The odds ratio for the synthetic platelets versus no injection is 13.3 with a 95% confidence interval of 1.24 to 143.

In early work with the synthetic platelets, a non-survival models was use. In this work, animals were maintained for up to 3 weeks post blast in both the nanoparticle control and synthetic platelets groups (n=7 per group). Only one animal died post 1 hour in the synthetic platelet 3 week group, and the death showed no signs of complications from particle administration such as signs of gasping, stroke, or other signs of blocked vessels. Rather, the animal became weak and was euthanized at one day post injury. Two animals in the nanoparticle control group failed to survive to the 3 week time point.

Preliminary histological analysis of the control and treatment groups demonstrated that active synthetic platelet treatments groups had lower levels of lung injury. The 20 μg/ml concentrations resulted in decreased levels of injury compared to the scrambled platelets and NovoSeven, which is a current clinical treatment for hemorrhaging. The trends in the lung injury data correlate well with the survival data with the reduction in injury (red blood cells) correlating with the increase in survival seen in the synthetic platelet group.

Biodistribution of the synthetic platelets and nanoparticle controls at 1 hour post blast (n=3) demonstrates that the particles are throughout the tissues with the greatest percentage being in the lungs, spleen, and liver. In the nanoparticle control group, approximately 10% are in each of the lungs. There are lower percentages in the synthetic platelet group, but the n for this work is still low and as the study continues, it will be interesting to see if there continue to be small amounts or if the numbers are more consistent with the controls. Biodistribution of the synthetic platelets or nanoparticle controls in sham (non-blasted) mice are similar to each other.

Clotting time plus clot formation time was reduced with synthetic platelets compared to blood alone or saline (n=2). The dose used for this study was 1.25 mg/ml which correlates well with the 20 mg/ml used in the blast model. The addition of saline appears to actually decrease clotting time compared to the blood-only control, suggesting that this addition may be activating the coagulation cascade however, the n is low and the study must be further validated. (p=0.4 for this date with n=2).

The shear modulus strength appears to recapitulate the shear modulus strength of the blood-only clot (p=0.76). The nanoparticle controls appear to reduce the shear modulus strength suggesting that the inactive peptide nanoparticles may disrupt the clot formation which could account for the slightly increased lethality with the nanoparticle controls.

Based on the findings related to this work, large animal (pig) liver injury studies were begun. Preliminary data suggests that synthetic platelets reduce blood loss in a large animal model and dosing for the pigs is far lower (as low as 3 mg/pig) than expected. For the rats, the optimal dose for the triblock was 20 mg/ml (0.5 ml of synthetic platelets injected.) For the quadblock version, it was 2.5 mg/ml (0.5 ml administered.)

### EXAMPLE 14

Intravenous administration of hemostatic nanoparticles that target activated platelets have been investigated by a number of groups with some promise and a range of challenges. RGD conjugated red blood cells (RBCs) called thromboerythrocytes showed promise in vitro but did not significantly reduce prolonged bleeding times in thrombocytopenic primates. Fibrinogen-coated albumin microparticles, “Synthocytes” and liposomes used by others carrying the fibrinogen γ chain dodecapeptide (HHLGGAKQAGDV) showed success in bleeding models in thrombocytopenic rabbits. However, Synthocytes were ineffective in treating bleeding in normal rabbits, and the liposomes do not appear to have yet been studied for this purpose.

From this work, several things are clear. First, if particles are too large or carry immunogenic materials, they may trigger non-specific thrombosis. Because the coagulation system is so complex, multiple bleeding models (and species) with functionally-directed outcomes, in concert with in vitro studies, are used to fully evaluate a potential therapy, as has been recognized by the FDA in a set of published guidelines for platelet substitutes 21. Prothrombotic potential, immunogenicity, and toxicity due to additives are among the safety criteria, and efficacy criteria is based on a battery of in vivo and in vitro tests.

Nanoparticle Preparation

A PLGA-PLL-PEG triblock polymer was synthesized using stepwise conjugation reactions, starting with PLGA (Resomer 50311) and poly(E-cbz-L-lysine) (PLL-cbz) PLL with carbobenzoxy-protected side amine side groups (Sigma P4510). This conjugation reaction was confirmed using UV-Vis to check for a signature triple peak corresponding to the cbz groups. After deprotecting the PLGA-PLL-cbz with HBr, the free amines on the PLL-NH3 were reacted with CDI-activated PEG in a 5:1 molar excess. The conjugated triblock copolymer PLGA-PLL-PEG (with CDI activated PEG endgroups) was dissolved to a concentration of 20 mg/ml in acetonitrile containing coumarin-6 (C6), a fluorescent dye is used to track the nanoparticles after injection (loaded at 1% w/w). This solution was added dropwise to a volume of stirring PBS, twice that of the acetonitrile. Precipitated nanoparticles form as the water-miscible solvent is displaced. The nanoparticles were then conjugated with GRGDS or the conservatively substituted GRADSP peptide and stir-hardened for 3 hours in a single step. Nanoparticles were then collected using the coacervate precipitation method described below.

One mass equivalent of dry poly(acrylic acid) (pAA) (Sigma, MW=1,800) was added to the stirring particle suspension. A 1% w/v solution of pAA was then added to the stilling suspension until flocculation occurred, approximately 10 ml. After 5 minutes, the flocculated nanoparticles were collected by centrifugation and rinsed 3 times. Nanoparticles were resuspended to approximately 10 mg/ml with deionized water, snap-frozen in liquid nitrogen and lyophilized for 3 days. Nanoparticles were resuspended to a concentration of 20 mg/ml in 1× PBS and briefly sonicated (VCX-130, Sonics & Materials, Inc.).

Nanoparticles were characterized for size distribution and polydispersity using dynamic light scattering (90Plus, Brookhaven Instruments Comoration) and scanning electron microscopy (Hitachi S4500). DLS data was represented as the effective diameter as calculated by the 90Plus software. SEM images were analyzed in ImageJ software. Successful conjugation of PLL, PEG and peptide ligands was confirmed using UV-spectroscopy, 1H-NMR and amino acid analysis HPLC (BioRad, Varian and Shimadzu respectively). 1H-NMR is performed with chloroform for analyzing the triblock structure and deuterated water to verify the PEG coronal shell 27. Amino acid analysis was performed by W. M. Keck Foundation Biotechnology Resource Laboratory (New Haven, Conn.).

Coagulation Assays

Coagulation assays, using Sprague Dawley rat blood, were performed as described above.

In Vivo Liver Injury Model

In order to assess the efficacy of the nanoparticles to augment survival in a lethal injury model, a liver injury model was adapted from Ryan et al. 28 and Holcomb et al. 29 and is described below. The injury model was approved and undertaken according to the guidelines set by Case Western Reserve University's institutional animal care and use committee. The main outcomes recorded for this study include survival at 1 hour and blood loss as measured with pre-weighed gauze.

Surgical procedure Sprague Dawley rats (225-275 g, Charles River) were anesthetized with intraperitoneal ketamine:xylazine (90:10 mg/kg, respectively). After 10 minutes, they were shaved and placed in a supine position on a heatpad. The abdomen was accessed and the medial lobe of the liver was marked with an arch radius 1.3 cm from the suprahepatic vena cava using a handheld cautery device. Once marked, the tail vein was exposed, and catheterized with a saline-flushed 24G×¾″ Excel Safelet Catheter. The medial liver lobe was then resected along the marked lines, the abdomen was closed with wound clips, and 0.5 cc bolus treatment solution was immediately administered followed by 0.2 cc saline flush to clear the catheter dead-volume.

The rats were allowed to bleed for 1 hour or until death, as confirmed by lack of both breathing and a palpable heartbeat. Before measuring blood loss, all rats were injected with a lethal dose of sodium pentobarbital (i.v.). The abdomen was then reopened and blood collected with pre-weighed gauze. The clot adherent to the liver was collected last as this usually caused additional bleeding to occur. The resected liver was weighed and fixed in 10% buffered formalin solution. Remaining liver, kidney, spleen, lungs and adherent clot were harvested and similarly preserved in 10% buffered formalin.

Procedure and Statistics

Treatments included no injection (n=3), saline (n=17), scrambled NPs (n=15), and hemostatic GRGDS-NPs (n=20). Particle treatments were resuspended to 20 mg/ml in PBS. The surgeon was blinded to the treatments and all blood loss measurements and death were independently recorded by a second person also blinded to the treatment. The no injection group (n=3) was included as a reference, but was not included in the statistics. ANOVA with Tukey comparisons was used to analyze blood loss data (Minitab). Survival was analyzed with a binomial logistic regression with chi-squared tests between odds-ratios (SAS). A power analysis based on preliminary studies suggested an n=15 per group for significance for survival data (alpha=0.05, beta=0.2, odds ratio=3).

Biodistribution

Liver, kidney, spleen, lung and adherent clots were harvested and lyophilized for the biodistribution assay. The dry weight of the whole organ was recorded and 100-200 mg of dry tissue was homogenized (Precellys 24) and incubated overnight in acetonitrile at 37 C. This dissolved any nanoparticles present in the tissue and left the C6 in the organic solvent solution. Tubes were then centrifuged at 15,000 g for 10 minutes to remove solid matter and supernatant was tested on the HPLC. Mobile phase was 80% acetonitrile, and 20% aqueous (8% acetic acid). Stationary phase was a Waters Symmetry C18 Column, 100A, 5 gm, 3.9 mm×150 mm with fluorescence detection (450/490 nm ex/em). Based on the known C6 loading and injection volume of particles, data is represented as percent (%) of particles injected. Imaging injury surface and adherent clots Resected portions of the liver were rinsed and placed directly on a high-resolution (1200 dpi) flatbed scanner (Cannon CanoScan LiDE 700F) to image the surface of the injury. Adherent clots, still attached to livers were fixed in 10% formalin, soaked overnight in sucrose, frozen and cryosectioned to 20-micron thickness. Sections were then stained with VectaShield DAPI to stain hepatocyte nuclei and imaged with an inverted fluorescence microscope (Zeiss Axio Observer.Z1). Several clots per group were fixed in 10% formalin, and dehydrated in serial steps with ethanol to prepare them for imaging with a scanning ACS Paragon Plus Environment electron microscope (SEM). These were then dried overnight in anhydrous hexamethyldisilazane and sputter coated. Samples were mounted and imaged with a Hitachi S4500 field emission SEM at 5k× magnification.

Results

Particle synthesis and characterization The PLGA-PLL-PEG triblock polymer is synthesized using stepwise conjugation reactions, starting with PLGA (Resomer 503H) and poly(E-cbz-L-lysine) PLL with carbobenzoxy-protected side amine side groups following Bertram et al. 22′ 23′ 313. Conjugation efficiency for this step is approximately—30-40% molar ratio PLL:PLGA, as determined by UV-vis. After deprotection of side groups, the free amines on the PLL are reacted with CDI-activated PEG. This PEG creates a hydrophilic shell around the nanoparticles that allow them to have a longer residence time in blood circulation. 11-1-NMR in deuterated chloroform and deuterated water is performed to verify the expected surface-pegylated structure. From the spectrum, percent pegylation is calculated to be 1:10 (PEG:PLGA) molar ratio. In deuterated water, the PEG peak becomes much larger in relation to the other peaks and confirms the PEG-coronal structure of the nanoparticles in an aqueous environment. The size and distribution of the nanoparticles cores (by SEM) and in the aqueous environment (by DLS) is homogenously distributed around 400 nm and 420 nm respectively. The increase in size from SEM to DLS can be accounted for by the hydration shell, created by the PEG arms. There appears to be a slight increase in size as a result of C6 loading (approximately 5-10%), with no significant change in size depending on the GRGDS or GRADSP peptide conjugated.

In vivo injury model development Following injury of the medial lobe, rats were administered either saline, scrambled (GRADSP), or hemostatic (GRGDS-conjugated) nanoparticles. Saline is used as the baseline control because the administration of fluids can impact bleeding. Based on our preliminary results, we found that resected liver mass and body mass were well-correlated with bleeding outcomes, and similar to Holcomb et al. 29, we chose to strictly adhere to inclusion criteria for rat body mass (225-275 g) and liver resection (0.8-1.2% of body mass). At the conclusion of the study, this inclusion criteria was found to reduce rat-to-rat variability based on body mass. However, liver resection mass was still significantly correlated with bleeding outcomes (p=0.0004). When resected liver mass and treatment are included in the ANOVA model, the treatment is still not significantly correlated with bleeding outcomes (p=0.113).

One of the most critical parts of this work was to determine whether administration of the nanoparticles led to improved survival following blunt trauma injury. Administration of the hemostatic, GRGDS nanoparticles significantly improves survival following the lethal liver injury. Specifically, the GRGDS-NPs increases the odds of survival to 80%. This is compared to 47% in the saline group (p=0.040, odds ratio (OR)=4.5, 95% CI 1.1-19.2) and 40% in the scrambled-NP group (p=0.019, OR=6, 95% CI 1.3-27.0). Administering the GRGDS-NPs almost doubles the chances of survival from this lethal injury. Blood loss We know from our previous work 22 that the GRGDS-NPs reduce bleeding. In this work, we measured blood loss through the weight change in gauze used to adsorb the blood in the body cavity at the end of the experiment. This method gives data on blood loss but lacks the fine resolution permitted in the previous study. Measuring total blood loss in this model is complicated by the impact of survival time. The rate of blood loss may be a better indicator of survival for this model, but since the injury model is maintained in the small, closed cavity of rats, blood loss could not be dynamically measured. Nonetheless, we saw a trend in blood loss that correlates with survival with the GRGDS-NPs exhibiting the least blood loss. This trend towards reduction in blood loss is not statistically significant (13=0.0552), but it suggests that the GRGDS-NPs are improving survival through mitigation of bleeding. There also appears to be a critical threshold around 35% blood volume loss, above which there is rapidly increasing proportion of mortality.

Imaging Injury Surface

To help validate that our GRGDS-NPs are targeting the injury site, and accumulating within the clot, we imaged the injury surface using several modalities including fluorescent microscopy and SEM. Nanoparticles loaded with the fluorescent compound coumarin-6 (C6) are found within the injury surface, integrated with the clot. The injury surface is also characterized using a flatbed scanner to help depict the nature of the injury. From visual observation of the injury during model development, it is apparent that the majority of bleeding occurs through the 2-4 major blood vessels that are transected in the medial lobe injury.

Biodistribution

For the GRGDS-NPs, 31.1% of the injected dose is found in the clot versus only 6.8% for the scrambled-NP group. Total recovery of the nanoparticles between the clot and organs tested was 53.7% and 29.6% for the GRGDS-NPs and scrambled-NP groups, respectively; the unrecovered proportion is most likely located in the shed blood, not actively participating in the clot, or remaining in plasma circulation. There was a relatively large percentage of nanoparticles found in the lungs for each group, 20.8% and 20.6% (GRGDS and Scrambled, respectively), and a small percentage found in the other organs tested (<2%).

In Vitro Coagulation Model

A dosing study was performed using rotational thromboelastometry (ROTEM), with citrated rat. In this assay, a 20 ill volume of PBS containing a varying concentration of nanoparticles was added to a 300 ill volume of blood immediately before starting the assay. In addition to saline, concentrations of nanoparticles tested included 0.625, 1.25, 2.5, 5.0, and 20 mg/ml for GRGDS and scrambled nanoparticle groups. In all concentrations tested in the scrambled group, the CT+CFT increased and the MCF decreased compared to saline. In GRGDS-NP 1.25 and 2.5 mg/ml concentrations, MCF increased. Similarly, the clotting time is decreased in 1.25 mg/ml, and 5.0 mg/ml groups, but was increased otherwise. This is indicative of a clot forming faster and thicker when treated with the nanoparticles at an optimal dose, approximately 73.5-294 gg/ml in the blood or a 5.2-20 mg/kg dose for a 250 g male rat, assuming 68.6 ml/kg blood volume 32.

Concentrations of 1.25 and 2.5 mg/ml concentrations were further investigated as these had the most favorable effects on clotting parameters. Arandomized block experimental method was used, with saline as the control for each test-block. The 2.5 mg/ml GRGDS-NP dose significantly reduced clotting time compared to saline controls (p=0.0437) and had a trend toward increasing MCF although the difference was not significant (n=3 rats, with triplicate measurements at each treatment-dose level). The 2.5 mg/ml GRGDS-NP dose significantly reduced clotting time compared to saline controls (p=0.0437) and had a trend toward increasing MCF although not statistically significant. Interestingly, the scrambled-NP groups also appeared to reduce clotting times and increase MCF, but the differences were not significantly different from either saline or GRGDS treatments.

Administration of hemostatic nanoparticles increased 1-hour survival Early intervention is critical to improve chances of survival following trauma, and we see the effects of early intervention in this work. For all groups tested, there was a window of 20 minutes, after which, the odds of survival improved, as well as a critical blood volume loss of approximately 35% blood volume, below which 95% of rats survived.

Nearly twice as many rats survive one hour with administration of the hemostatic nanoparticles compared to controls. This result is statistically significant and clinically tremendous. We have seen previously that these hemostatic nanoparticles are stable at room temperature and reduce bleeding in a controlled injury model, but one of the major questions was whether this reduction in blood loss would impact survival in lethal trauma models of bleeding. The liver injury model is one of the most reproducible and comparable in the field 28′ 29′ 33. Seeing an almost two fold increase in survival with the GRGDS-NPs confirms that they not only reduce bleeding but do so at a level that impacts survival in the critical prehospital window.

There is a 4.5-fold higher amount of GRGDS-NPs found in the adherent liver clot compared to the scrambled-NP group, with very small quantities of nanoparticles found in the kidney, spleen and uninjured liver, confirming their injury-targeting capability. Nearly 20% of injected nanoparticles have been found in the lungs regardless of the treatment group. While some basal level of nanoparticles in the lungs is expected due to the pulmonary perfusion still present in the organ at the time of collection, previous studies in naïve rats estimate this to account for only 5-10% of the injected dose 22. These findings may indicate that the nanoparticles could be accumulating in thromboemboli in the lungs, concomitant with the massive hemonhagic nature of this injury model 34. However, it is of particular interest to note that survival does not appear to be deleteriously impacted—rather the opposite. It therefore reasons to argue that these thrombi are also present in the saline control, and may be present as microemboli that may not have any clinical presentation 34′ 35. Future studies may be aimed at assessing the risk of particle aggregation in the lungs and determining what functional impacts they may have, for example, by monitoring lung perfusion, tissue oxygenation, or blood gas levels. The ease of intravenous administration of these nanoparticles, coupled with their effective injury-targeting without deleterious functional outcomes bodes well for translation of this therapy to the clinic.

A trend toward reduction in blood loss was observed with the functionalized treatment versus controls. However, the methods for blood collection in trauma models in rats are limited, and the sensitivity is modest at best. Therefore, it is not surprising that no differences between the groups in this area to statistical significance could be resolved. While this model is not acutely sensitive to differences in blood loss, the trend regarding blood loss correlates well with the survival outcomes, the key point of this study.

The effect of nanoparticles on clotting times was dose dependent and an efficient dose tested was the 2.5 mg/ml group, corresponding to a blood concentration of 147 μg/ml (particle mass/blood volume). Based on in vitro findings, where the nanoparticles reduce clotting time and tend to increase clot firmness, it was hypothesize that for increased survival, more rapid clot formation and increase in clot strength gave rise to reduction in blood loss and increase in survival.

What is claimed is:

1. A temperature stable nanoparticle comprising a core, a water soluble polymer and a peptide, the water soluble polymer attached to the core at a first terminus of the water soluble polymer, the peptide attached to a second terminus of the water soluble polymer, the peptide comprising an RGD amino acid sequence, the water soluble polymer of having sufficient length to allow binding of the peptide to glycoprotein IIb/IIIa (GPIIb/IIIa).

2. The nanoparticle of claim 1 having a melting temperature over 35° C.

3. The nanoparticle of claim 1 or 2 having a spheroid shape and a diameter of less than 1 micron.

4. The nanoparticle of claim 3 having a diameter between 0.1 micron and 1 micron.

5. The nanoparticle of claim 1 or 2 which is non-spheroid.

6. The nanoparticle of claims 5 which is a rod, fiber or whisker.

7. The nanoparticle of claim 6 with an aspect ratio length to width of at least 3.

8. The nanoparticle of any of claims 1-7 which is stable at room temperature for at least 14 days.

9. A plurality of nanoparticles, each nanoparticle according to any of claims 1-8, wherein nanoparticles in the plurality have an average diameter between 0.1 micron and 1 micron.

10. The plurality of nanoparticles of claim 9 wherein greater than 75% of all nanoparticles have a diameter between 0.1 micron and 1 micron.

11. The nanoparticle of any of claims 1-8 wherein the core is crystalline polymer.

12. The nanoparticle of claim 11 wherein the core is a single polymer, a block copolymer, a triblock copolymer or a quadblock polymer.

13. The nanoparticle of any of claims 1-8, 11 and 12 wherein the core comprises PLGA, PLA, PGA, (poly (ε-caprolactone) PCL, PLL or combinations thereof.

14. The nanoparticle of any of claims 1-8, and 11-13 wherein the core is biodegradable.

15. The nanoparticle of any of claims 1-8 wherein the core is solid.

16. The nanoparticle of any of claims 1-8 and 15 wherein the core is non-biodegradable.

17. The nanoparticle of any of claims 1-8, 15 and 16 wherein the core is a material selected from the group consisting of gold, silver, platinum, aluminum, palladium, copper, cobalt, indium, nickel, ZnS, ZnO, Ti, TiO₂, Sn, SnO₂, Si, SiO₂, Fe, Fe⁺⁴, steel, cobalt-chrome alloys, Cd, CdSe, CdS, and CdS, titanium alloy, AgI, AgBr, HgI₂, PbS, PbSe, ZnTe, CdTe, In₂S₃, In₂Se₃, Cd₃P₂, Cd₃As₂, InAs, GaAs, cellulose or a dendrimer structure.

18. The nanoparticle of any of claims 1-8 and 11-17 wherein the water soluble polymer is selected from the group consisting of polyethylene glycol (PEG), branched PEG, polysialic acid (PSA), carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG), polyoxazoline, poly acryloylmorpholine, polyvinyl alcohol (PVA), polycarboxylate, polyvinylpyrrolidone, polyphosphazene, polyoxazoline, polyethylene-co-maleic acid anhydride, polystyrene-co-maleic acid anhydride, poly(1-hydroxymethylethylene hydroxymethylformal) (PHF), 2-methacryloyloxy-2′-ethyltrimethylammoniumphosphate (MPC), polyethylene glycol propionaldehyde, copolymers of ethylene glycol/propylene glycol, monomethoxy-polyethylene glycol, carboxymethylcellulose, polyacetals, poly-1,3-dioxolane, poly-1,3,6-trioxane, ethylene/maleic anhydride copolymer, poly (β-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll or dextran and combinations or mixtures thereof.

19. The nanoparticle of claim 18 wherein the water soluble polymer is PEG.

20. The nanoparticle of claim 19 wherein the PEG has an average molecular weight between 100 Da and 10,000 Da.

21. The nanoparticle of claim 19 wherein PEG has an average molecular weight of at least about 100.

22. The nanoparticle of any of claims 1-8 and 11-21 wherein the peptide comprises a sequence selected from the group consisting of RGD, RGDS, GRGDS, GRGDSP, GRGDSPK, GRGDN, GRGDNP, GGGGRGDS, GRGDK, GRGDTP, cRGD, YRGDS or variants thereof.

23. The nanoparticle of any of claims 1-8 and 11-22 wherein the RGD peptide is in a tandem repeat.

24. The nanoparticle of any of claims 1-8 and 11-23 comprising 1, 2, 3, 4, 5, 6, 7, 8, 9, 10 or more copies of the RGD peptide.

25. The nanoparticle of any of claims 1-8, and 11-22 comprising multiple copies of the RGD peptide.

26. The nanoparticle of claim 25 wherein all copies of the RGD peptide are the same.

27. The nanoparticle of claim 25 wherein two copies of the RGD peptide have different sequences.

28. The nanoparticle of any of claims 1-8 and 11-27 wherein the water soluble polymer is attached to the core at a molar ratio of 0.1:1 to 1:10 or greater.

29. The nanoparticle of any of claims 1-8 and 11-27 further comprising a therapeutic compound.

30. The nanoparticle of claim 29 wherein the therapeutic compound is hydrophobic.

31. The nanoparticle of claim 29 wherein the therapeutic compound is hydrophilic.

32. The nanoparticle of any of claims 29-31 wherein the therapeutic compound is covalently attached to the nanoparticle, non-covalently associated with the nanoparticle, associated with the nanoparticle through electrostatic interaction, or associated with the nanoparticle through hydrophobic interaction.

33. The nanoparticle of any of claims 29-31 wherein the therapeutic compound is a growth factor, a cytokine, a steroid, or a small molecule.

34. The nanoparticle of any of claims 29-32 wherein the therapeutic compound is a anti-cancer compound.

35. A pharmaceutical composition comprising the nanoparticle of any of claims 1-8 and 11-38.

36. The pharmaceutical composition of claim 35 in an intravenous administration formulation.

37. The pharmaceutical composition of claim 35 which is lyophilized or a powder.

38. A method of treating an condition in an individual comprising the step of administering the nanoparticle of any of claims 1-8 and 11-38 to a patient in need thereof in an amount effective to treat the condition.

39. The method of claim 38 wherein the individual has a bleeding disorder.

40. The method of claim 39 wherein the nanoparticle is administered in an amount effective to reduce bleeding time by more than 15% compared to no administration or administration of saline.

41. The method of claim 39 or 40 wherein the bleeding disorder is a symptom of a clotting disorder, thrombocytopenia, a wound healing disorder, trauma, blast trauma, a spinal cord injury or hemorrhaging.
